(19) United States
(12) Patent Application Publication
(10) Pub. No.: US 2014/0296446 A1
(43) Pub. Date: Oct. 2, 2014

(54) Modifying Polymeric Materials By Amines

(71) Applicant: Dow Corning Corporation, MIDLAND, MI (US)

(72) Inventors: Michael Wolfgang Backer, Mainz (DE); Thomas Chaussee, Fontaines Saint Martin (FR); Olivier Debever, Lembeek (BE)

(21) Appl. No.: 14/362,665

(22) PCT Filed: Dec. 12, 2012
(86) PCT No.: PCT/EP2012/074731

§ 371 Date: Jun. 4, 2014

(30) Foreign Application Priority Data
Dec. 8, 2011 (GB) 1121132.3

(51) Int. Cl. C08F 8/32; C07D 203/08

(52) U.S. Cl. CPC C08F 8/32; C07D 203/08
USPC 525/375; 548/964

(57) ABSTRACT

This invention relates to the modification of polymeric materials containing reactive carbon-to-carbon unsaturation and to amines, including aziridines, which are used in such modification. A polymeric material containing carbon-to-carbon bonds can be modified by crosslinking or to make it susceptible to crosslinking.

This invention relates to the modification of polymeric materials containing reactive carbon-to-carbon unsaturation and to amines, including piperazines and aziridines, which are used in such modification. A polymeric material containing carbon-to-carbon bonds can be modified by crosslinking or to make it susceptible to crosslinking.

Many of the amines, including piperazines and aziridines, which are used in the modification of materials containing carbon-to-carbon unsaturation are new compounds. Thus the invention also relates to substituted piperazines and to their preparation, to substituted aziridines and to their preparation, and to other substituted amines and to their preparation.

An article in Russian Journal of Applied Chemistry; Volume 82, Issue 5, Pages 928-930; Journal 2009; by V. M. Farzaliev, M. T. Abbasova, A. A. Ashurova, G. B. Babaeva, N. P. Ladokhina and Ya. M. Kerimova describes the preparation of bis(alkoxymethyl)piperazines by condensation of piperazine with formaldehyde and aliphatic alcohols.

A process according to another aspect of the invention for modifying a polymeric material containing carbon-to-carbon unsaturation is characterised in that the polymeric material is treated with a compound (II) containing in its molecule at least two moieties of the formula —OC(O)-(Az)-J wherein Az represents an aziridine ring bonded to the group J through its nitrogen atom; and J represents a hydrocarbyl or substituted hydrocarbyl group having 1 to 20 carbon atoms or can contain another Az.

The amine compounds (II) of the invention are capable of crosslinking a polymeric material containing carbon-to-carbon unsaturation. We believe that upon heating, for example to the temperatures used in elastomer processing, the etheramine moiety of (II) forms a very reactive species which reacts with the C═C bonds present in the polymeric material through [2+3] cycloaddition.

Thus in one process according to the invention the polymeric material and the amine compound (II) are heated together at a temperature of 120to 200° C., whereby the polymeric material is crosslinked by the substituted aziridine.

In an alternative process according to the invention the polymeric material and the amine compound (II) are mixed at a temperature of 0 to 120° C. and subsequently heated at a temperature of 120to 200° C. to crosslink the polymeric material. When mixing at an elevated temperature below 120° C., there may be some modification of the polymeric material which can be detected via infra-red spectroscopy, for example at least some of the amine compound (II) may be bonded to the polymeric material without substantial crosslinking.

The aziridine compound (II) contains in its molecule at least two moieties of the formula —OC(O)-(Az)-J wherein Az represents an aziridine ring bonded to the group J through its nitrogen atom; and J represents a hydrocarbyl or substituted hydrocarbyl group having 1 to 20 carbon atoms. The compound (II) can in general be prepared by reacting a 2,3-dibromopropionate containing in its molecule at least two moieties of the formula —OC(O)—CHBr—CH₂Br with an amine of the formula J-NH₂

The 2,3-dibromopropionates containing at least two moieties of the formula —OC(O)—CHBr—CH₂Br can be prepared by the reaction of an acrylate containing at least two acrylate groups of the formula —OC(O)—CH═CH₂ with bromine at ambient temperature or below.

The compound (II) can for example be of the formula —OC(O)-Az)-J-Az-(O)C—O—. The invention also includes a process for the preparation of such an aziridine compound (II) by reacting a 2,3-dibromopropionate ester with J being a diamine with a hydrocarbyl, or substituted hydrocarbyl spacer e.g. ethylene diamine, polyether(diamines).

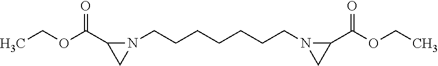 [SMILES: CCOC(=O)C1CN1CCCCCCCN1CC1C(=O)OCC]

The compound (II) can for example be of the formula Q(—OC(O)-(Az)-J)ₓ wherein x=2 to 6; and Q is the residue of a polyol having at least x hydroxyl groups. Such compounds are new compounds and form part of this invention. The invention also includes a process for the preparation of such an aziridine compound (II) by reacting a polyol 2,3-dibromopropionate ester of the formula Q(—OC(O)—CHBr—CH₂Br)ₓ wherein x=1 to 6 and Q is the residue of a polyol having at least x hydroxyl groups is reacted with an amine of the formula J-NH₂ wherein J represents a hydrocarbyl or substituted hydrocarbyl group having 1 to 20 carbon atoms. The polyol 2,3-dibromopropionate ester can be prepared from a polyol polyacrylate by reaction with bromine.

Examples of polyol polyacrylates that can be brominated and reacted with an alkoxysilylalkylamine include diacrylates such as ethyleneglycol diacrylate, di- and triethyleneglycol diacrylates and polyethyleneglycol diacrylates of varying chain lengths, propyleneglycol diacrylate, di- and tripropyleneglycol diacrylate and polypropyleneglycol diacrylates of varying chain lengths, butanediol-1,3- and -1,4-diacrylates, neopentylglycol diacrylate, hexanediol-1,6-diacrylate, isosorbide diacrylate, 1,4-cyclohexanedimethanol diacrylate, bisphenol-A-diacrylate and the diacrylates of hydroquinone, resorcinol lengthened with ethylene oxide and propylene oxide, triacrylates such as trimethylolpropane triacrylate, glycerol triacrylate, trimethylolethane triacrylate, 2-hydroxymethylbutanediol-1,4-triacrylate, and the triacrylates of glycerol, trimethylolethane or trimethylolpropane lengthened with ethylene oxide- or propylene oxide., and higher polyol acrylates such as pentaerythritol tetraacrylate and di-pentaerythritol hexaacrylate.

One example of a preferred compound (II) of the formula Q(—OC(O)-(Az)-J)ₓ has the formula

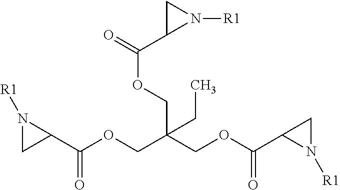

wherein R1 represents an alkyl group having 1 to 6 carbon atoms. This can be prepared by the reaction of trimethylolpropane triacrylate with bromine, and reacting the 2,3-dibromopropionate ester produced with an alkylamine.

The aziridine compound (II) can alternatively be a metal carboxylate of the formula M(—OC(O)-(Az)-J)ₘ, wherein M represents a metal ion of valence m; Az represents an aziridine ring bonded to the group J through its nitrogen atom; and J represents a hydrocarbyl or substituted hydrocarbyl group having 1 to 20 carbon atoms. Such aziridine metal carboxylate compounds are new compounds and form part of this invention.

The invention also includes a process for the preparation of such an aziridine metal carboxylate compound (II) by reacting a metal 2,3-dibromopropionate salt of the formula:

M(—OC(O)—CHBr—CH₂Br)₂

wherein M represents a divalent metal ion, with an amine of the formula J-NH₂ wherein J represents a hydrocarbyl or substituted hydrocarbyl group having 1 to 20 carbon atoms.

The metal 2,3-dibromopropionate salt of the formula:

M(—OC(O)—CHBr—CH₂Br)₂

can be prepared from the corresponding metal diacrylate by reaction with bromine.

A preferred divalent metal M is zinc. Alternative divalent metals include magnesium, copper and iron. One example of a preferred aziridine metal carboxylate compound (II) has the formula

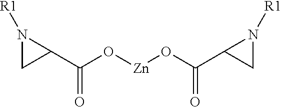

wherein R1 represents an alkyl group having 1 to 6 carbon atoms. This can be prepared by reacting zinc diacrylate with bromine, and reacting the zinc di(2,3-dibromopropionate) produced with an alkylamine.

The polymeric material containing carbon-to-carbon unsaturation can for example be a diene rubber. The diene elastomer can for example be natural rubber. The diene elastomer can alternatively be a synthetic polymer which is a homopolymer or copolymer of a diene monomer (a monomer bearing two double carbon-carbon bonds, whether conjugated or not). Preferably the elastomer is an “essentially unsaturated” diene elastomer, that is a diene elastomer resulting at least in part from conjugated diene monomers, having a content of members or units of diene origin (conjugated dienes) which is greater than 15 mol %. More preferably it is a “highly unsaturated” diene elastomer having a content of units of diene origin (conjugated dienes) which is greater than 50 mol %.

The diene elastomer can for example be:

(a) any homopolymer obtained by polymerization of a conjugated diene monomer having 4 to 12 carbon atoms;

(b) any copolymer obtained by copolymerization of one or more dienes conjugated together or with one or more vinyl aromatic compounds having 8 to 20 carbon atoms;

(c) a ternary copolymer obtained by copolymerization of ethylene, of an α-olefin having 3 to 6 carbon atoms with a non-conjugated diene monomer having 6 to 12 carbon atoms, such as, for example, the elastomers obtained from ethylene, from propylene with a non-conjugated diene monomer of the aforementioned type, such as in particular 1,4-hexadiene, ethylidene norbornene or dicyclopentadiene;

(d) a copolymer of isobutene and isoprene (butyl rubber), and also the halogenated, in particular chlorinated or brominated, versions of this type of copolymer.

Suitable conjugated dienes include 1,3-butadiene, 2-methyl-1,3-butadiene, 2,3-di(Ci-C5 alkyl)-1,3-butadienes such as, for instance, 2,3-dimethyl-1,3-butadiene, 2,3-diethyl-1,3-butadiene, 2-methyl-3-ethyl-1,3-butadiene, 2-methyl-3-isopropyl-1,3-butadiene, an aryl-1,3-butadiene, 1,3-pentadiene and 2,4-hexadiene.

Suitable vinyl aromatic compounds are, for example, styrene, ortho-, meta- and para-methylstyrene, the commercial mixture “vinyltoluene”, para-tert.-butylstyrene, methoxystyrenes, chlorostyrenes, vinylmesitylene, divinylbenzene and vinylnaphthalene. The copolymers may contain between 99% and 20% by weight of diene units and between 1% and 80% by weight of vinyl aromatic units. The elastomers may have any microstructure, which is a function of the polymerization conditions used, in particular of the presence or absence of a modifying and/or randomizing agent and the quantities of modifying and/or randomizing agent used. The elastomers may for example be block, statistical, sequential or microsequential elastomers, and may be prepared in dispersion or in solution; they may be coupled and/or starred or alternatively functionalized with a coupling and/or starring or functionalizing agent. Examples of preferred block copolymers are styrene-butadiene-styrene (SBS) block copolymers and styrene-ethylene/butadiene-styrene (SEBS) block copolymers.

The elastomer can be an alkoxysilane-terminated diene polymer or a copolymer of the diene and an alkoxy-containing molecule prepared via a tin coupled solution polymerization.

The amine compound of formula (II) can be used as the only crosslinking agent for the diene elastomer or can be used in conjunction with a known curing agent for the elastomer composition, for example be a conventional sulfur vulcanizing agent.

The amine compound of formula (II), particularly a substituted piperazine, can alternatively be incorporated in a diene elastomer composition, particularly a natural rubber composition used in tyres, as an anti-reversion agent. An anti-reversion agent is an agent used in natural rubber to “heal” and cure the rubber while it is degrading with high temperature (160° C.). Heat durability of a tire tread is often a factor for vehicular tires intended to be driven at relatively high speeds. Heat is inherently generated within a tire tread rubber compound as the tire is driven at relatively high speeds resulting in a temperature rise within the tire tread itself.

It is desired to reduce the rate of temperature rise within a sulfur cured tire tread rubber composition with an attendant increase in its heat durability. Incorporation of an amine compound of formula (II) particularly a substituted piperazine, in the tread rubber composition retards the rate of temperature rise within the tread rubber composition.

When the amine compound of formula (II) is incorporated in a sulfur cured tire tread rubber composition as an anti-reversion agent, the amine compound of formula (I) can for example be added with the vulcanization system. The rubber compositions are preferably produced using the conventional two successive preparation phases of mechanical or thermo-mechanical mixing or kneading (“non-productive” phase) at high temperature, followed by a second phase of mechanical mixing (“productive” phase) at lower temperature, typically less than 110° C., for example between 40° C. and 100° C., during which the vulcanization system is incorporated. If the amine compound of formula (I) is incorporated in the rubber composition at this lower temperature, it does not act significantly as a crosslinking agent during production of the cured rubber, but remains in the rubber composition to act as an anti-reversion agent.

The polymeric material containing carbon-to-carbon unsaturation can alternatively be an organopolysiloxane containing alkenyl groups. Examples of alkenyl groups of the organopolysiloxane include vinyl, allyl, butenyl, pentenyl, hexenyl, and heptenyl groups, of which vinyl groups are preferred. Silicon-bonded organic groups other than alkenyl groups contained in the organopolysiloxane may be exemplified by methyl, ethyl, propyl, butyl, pentyl, hexyl, or similar alkyl groups; phenyl, tolyl, xylyl, or similar aryl groups; or 3-chloropropyl, 3,3,3-trifluoropropyl, or similar halogen-substituted groups. Preferably, the groups other than alkenyl groups are methyl groups and optionally phenyl groups.

For many uses it is preferred that the major part of the organopolysiloxane has a predominantly linear molecular structure, such as a polydiorganosiloxane. The organopolysiloxane can for example comprise an α,ω-vinyldimethylsiloxy polydimethylsiloxane, an α,ω-vinyldimethylsiloxy copolymer of methylvinylsiloxane and dimethylsiloxane units, and/or an α,ω-trimethylsiloxy copolymer of methylvinylsiloxane and dimethylsiloxane units.

The organopolysiloxane can additionally or alternatively comprise a branched organopolysiloxane containing alkenyl units. Such a branched organopolysiloxane can for example comprise ViSiO_{3/2} (where Vi represents vinyl), CH₃SiO_{3/2} and/or SiO_{4/2} branching units with (CH₃₎₂ViSiO_{1/2} and/or (CH₃₎₃SiO_{1/2} and optionally CH₃ViSiO_{2/2} and/or (CH₃₎₂SiO_{2/2} units, provided that at least one vinyl group is present. A branched organopolysiloxane can for example consist of (i) one or more Q units of the formula (SiO_{4/2}) and (ii) from 15 to 995 D units of the formula R^{b}₂SiO_{2/2}, which units (i) and (ii) may be inter-linked in any appropriate combination, and M units of the formula R^{a}R^{b}₂SiO_{1/2}, wherein each R^{a} substituent is selected from the group consisting of an alkyl group having from 1 to 6 carbon atoms, an alkenyl group having from 1 to 6 carbon atoms and an alkynyl group having from 1 to 6 carbon atoms, at least three Ra substituents in the branched siloxane being alkenyl or alkynyl units, and each R^{b} substituent is selected from the group consisting of an alkyl group having from 1 to 6 carbon atoms, an alkenyl group having 2 to 6 carbon atoms, an aryl group, an alkoxy group, an acrylate group and a methacrylate group, as described in U.S. Pat. No. 6,806,339.

The polyorganosiloxane can for example have a viscosity of at least 100 mPa·s at 25° C., preferably at least 300 mPa·s, and may have a viscosity of up to 90000 mPa·s, preferably up to 70000 mPa·s.

Organopolysiloxanes containing alkenyl groups are used for example in release coating compositions for paper and other substrates, and in liquid silicone rubber compositions used for coating air bags and for other applications. The amine compound of formula (II), particularly a substituted piperazine, can be used as all or part of the crosslinking agent in such compositions.

### EXAMPLES

### Prophetic Example 1

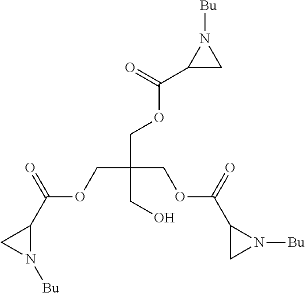

Detailed synthesis of pentaerythritol-triaziridine cross-linker. A 1 L two necked round bottom flask, fitted with a condenser, nitrogen sweep and magnetic stirrer, will be charged with 29.8 g n-butylamine, 97.6 g triethylamine and 400 ml toluene and inerted with nitrogen. To this ice-cold mixture will be added drop-wise a solution of 100.0 g pentaeythritol tri-(2,3-dibromopropionate) in 160 ml toluene. Mixture will be refluxed for 6 hours and solids filtered off over diatomaceous earth. Solvent and volatiles will be removed in vacuo affording the triaziridine cross-linker. Formation of the aziridine ring will be confirmed by nuclear magnetic resonance spectroscopy.

1. A process for modifying a polymeric material containing carbon-to-carbon unsaturation, characterised in that the polymeric material is treated with a compound (II) containing in its molecule at least two moieties of the formula —OC(O)-(Az)-J wherein Az represents an aziridine ring bonded to the group J through its nitrogen atom; and J represents a hydrocarbyl or substituted hydrocarbyl group having 1 to 20 carbon atoms.

2. A process according to claim 1, characterised in that the compound (II) has the formula Q(—OC(O)-(Az)-J)ₓ wherein x=2 to 6; and Q is the residue of a polyol having at least x hydroxyl groups.

3. A process according to claim 1, characterised in that the compound (II) has the formula M(—OC(O)-(Az)-J)ₘ wherein M represents a metal ion of valence m, preferably a divalent metal ion.

4. A process according to claim 1 wherein the polymeric material and the compound (II) are heated together at a temperature of 120 to 200° C., whereby the polymeric material is crosslinked by the compound (II).

5. A process according to claim 1 wherein the polymeric material and the compound (II) are mixed at a temperature of 0 to 120° C. and subsequently heated at a temperature of 120 to 200° C. to crosslink the polymeric material.

6. A process according to claim 1, characterised in that the polymeric material is a diene rubber.

7. A process according to claim 1, characterised in that the polymeric material is an organopolysiloxane containing alkenyl groups.

8. An aziridine compound having the formula Q(—OC(O)-(Az)-J)ₓ wherein x=1 to 6; Q is the residue of a polyol having at least x hydroxyl groups: Az represents an aziridine ring bonded to the group J through its nitrogen atom; and J represents a hydrocarbyl or substituted hydrocarbyl group having 1 to 20 carbon atoms.

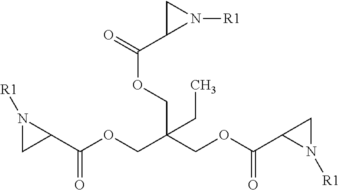

9. A process for the preparation of an aziridine compound as claimed in claim 8, characterised in that a polyol 2,3-dibromopropionate ester of the formula:
Q(—OC(O)—CHBr—CH2Br)ₓ
wherein x=1 to 6 and Q is the residue of a polyol having at least x hydroxyl groups is reacted with an amine of the formula J-NH₂ wherein J represents a hydrocarbyl or substituted hydrocarbyl group having 1 to 20 carbon atoms.

10. An aziridine compound having the formula M(—OC(O)-(Az)-J)₂ wherein M represents a divalent metal ion; Az represents an aziridine ring bonded to the group J through its nitrogen atom; and J represents a hydrocarbyl or substituted hydrocarbyl group having 1 to 20 carbon atoms.

11. An aziridine compound according to claim 11 having the formula: 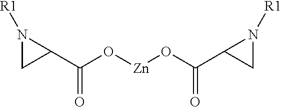
wherein R1 represents an alkyl group having 1 to 6 carbon atoms.

12. A process for the preparation of an aziridine compound as claimed in claim 12, characterised in that a metal 2,3-dibromopropionate salt of the formula:
M(—OC(O)—CHBr—CH₂Br₂
wherein M represents a divalent metal ion is reacted with an amine of the formula J-NH₂ wherein J represents a hydrocarbyl or substituted hydrocarbyl group having 1 to 20 carbon atoms.

13. (canceled)

14. An aziridine compound according to claim 8 having the formula: 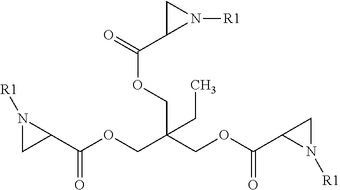
wherein R1 represents an alkyl group having 1 to 6 carbon atoms.
